(19) United States
(12) Patent Application Publication
(10) Pub. No.: US 2008/0306485 A1
(43) Pub. Date: Dec. 11, 2008

(54) INSTRUMENTS FOR KNEE SURGERY AND METHOD UF USE

(75) Inventors: Thomas M. Coon, Redding, CA (US); E. Marlowe Goble, Alta, WY (US); Alfred J. Tria, JR., Princeton, NJ (US); Warren Scott Gareiss, Columbia City, IN (US); Robert A. Hodorek, Warsaw, IN (US); Daniel F. Justin, Logan, UT (US); Donald M. Smucker, Perrysburg, OH (US); Richard R. Van Zile, Bryan, OH (US); Toby N. Farling, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 12/195,210

(22) Filed: Aug. 20, 2008

Related U.S. Application Data
(62) Division of application No. 10/356,404, filed on Feb. 3, 2003.
(60) Provisional application No. 60/383,347, filed on May 24, 2002.

(51) Int. Cl. A61B 17/60

(52) U.S. Cl. 606/88

(57) ABSTRACT

The present invention comprises a set of instruments and a method for their use in preparing a knee joint to receive knee implants. The inventive instruments and method are generally suitable for knee joint surgery. Furthermore, they include features that make them suitable for performing minimally invasive knee surgery.

### CROSS-REFERENCE TO RELATED APPLICATIONS

This application claims the benefit of U.S. Provisional Application No. 60/383,346, filed May 24, 2002.

### FIELD OF THE INVENTION

The present invention relates to methods and instruments for performing total knee arthroplasty.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is a perspective view of the lateral side of a knee.

FIG. 2 is a perspective view of a lateral compartment formed in the knee of FIG. 1.

FIG. 3 is a perspective view of a tibial template being mounted on a tibia of the knee of FIG. 1.

FIG. 4 is a front elevation view of the template of FIG. 3.

FIG. 5 is a side elevation view of the template of FIG. 3.

FIG. 6 is a perspective view of a tibial guide mounted on the tibia of FIG. 3.

FIG. 7 is a perspective view of an alternative embodiment of a guide system for mounting on a tibia.

FIG. 8 is a perspective view of the guide system of FIG. 7 mounted on a tibia.

FIG. 9 is a top plan view of the guide system of FIG. 7 mounted on a tibia.

FIG. 10 is a front elevation view showing a mechanical axis and an anatomic axis of a femur.

FIG. 11 is a perspective view of a guide wire being advanced toward the distal end of a femur.

FIG. 12 is a front elevation view of the guide wire shown in FIG. 11 disposed within the femur.

FIG. 13 is a perspective view of a positioning guide having the guide wire of FIG. 12 attached thereto.

FIG. 14 is a perspective view of an alignment pin.

FIG. 15 is a perspective view of a template positioned on a pair of alignment pins like that shown in FIG. 14.

FIG. 16 is a perspective view of the template shown in FIG. 15 having a cut guide mounted thereon.

FIG. 17 is an exploded perspective view of an alternative embodiment of a modular template.

FIG. 18 is a perspective view of the template of FIG. 18 with a distal cut stylus installed.

FIG. 19 is a perspective view of the template of FIG. 18 with a distal femoral cut guide installed.

FIG. 20 is a perspective view of the template of FIG. 18 with an anterior femoral cut guide installed.

FIG. 21 is a perspective view of the template of FIG. 18 with a posterior femoral cut guide installed.

FIG. 22 is a side elevation view of a set guide for use on a femur.

FIG. 23 is a schematic representation of mounting the set guide shown in FIG. 23 to a femur.

### DETAILED DESCRIPTION OF THE PREFERRED EMBODIMENTS

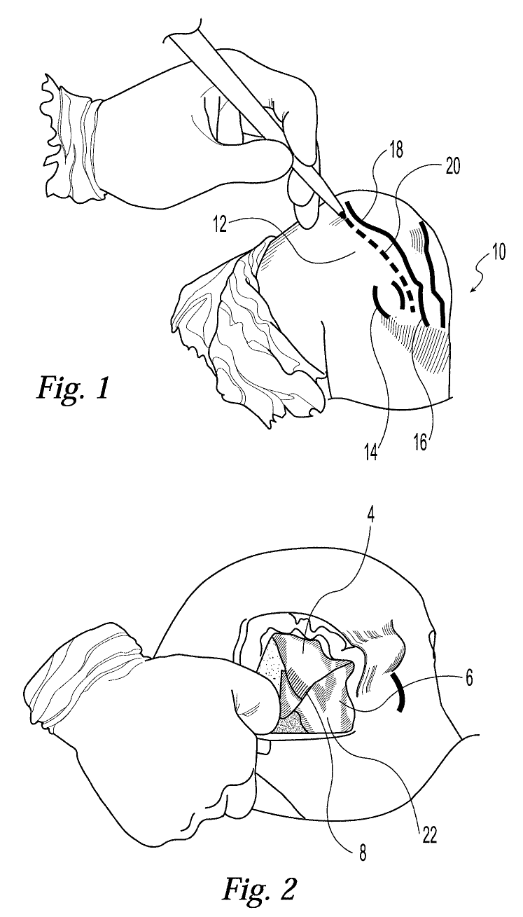

The present invention relates to methods and corresponding instruments for performing minimally invasive total knee arthroplasty. By way of example and not by limitation, depicted in FIG. 1 is a knee 10 having a lateral side 12. The knee 10 is flexed to about 90 degrees and marks are made on the skin over select areas. Specifically, marks 14 are made over Gerdy's tubercle, marks 16 are made over the tibial tuberosity, and marks 18 are made over the lateral border of the patella.

An incision, marked by a dashed line 20, is made beginning at the mid-portion of the patella and just lateral to the patellar border and extending along the lateral parapatellar region distally to the tibial tuberosity. The incision passes between the tibial tuberosity and Gerdy's tubercle. The lateral retinaculum is divided in line with the skin incision.

The lateral edge of the distal incision is now elevated from the bone at the tibia until Gerdy's tubercle is encountered. At this point Gerdy's tubercle is elevated, such as with a curved ½-inch osteotome, leaving approximately 2 mm of bone thickness to the illiotibial band insertion at Gerdy's tubercle. Subperiosteal dissection is then continued laterally along the proximal tibia to the posterior lateral corner. A retractor is put in place which extends around the posterolateral corner to a point midway between the posterior cruciate ligament and the posterolateral corner.

The anterior half of the lateral meniscus is excised, and a portion of the fat pad is also excised to give full visualization of the lateral compartment. The tissue just proximal to the tibial tuberosity is subperiosteally dissected from the tibial tuberosity to the joint line. Subperiosteal dissection is then carried out medially, elevating the capsule and soft tissue from the joint line to a point approximately 8 mm distal to the joint. This is elevated around the anteromedial corner, and a retractor is put in place containing the patella tendon and the anteromedial capsule. As depicted in FIG. 2, the exposure now reveals a lateral compartment 22, both anteriorly and laterally, wherein the distal end of a femur 4, proximal end of a tibia 6, and a joint line 8 formed therebetween are exposed.

Optionally, the lateral epicondyle can now be osteotomized. A curved ⅜-inch osteotome is utilized to elevate the base of the lateral epicondyle beginning anteriorly and extending distally to the edge of the articular surface and then elevating it as a Greenstick fracture by leaving the final attachment at the posterior border of the epicondyle. This is elevated sharply, and the attached tendons are retracted posterolaterally to expose the lateral joint and allow for opening with varus stress to expose the posterior horn of the lateral meniscus and the posterior cruciate ligament.

This optional osteotomy is utilized to give exposure and access to the posterior cruciate to protect this structure when the tibial cut is made from lateral to medial.

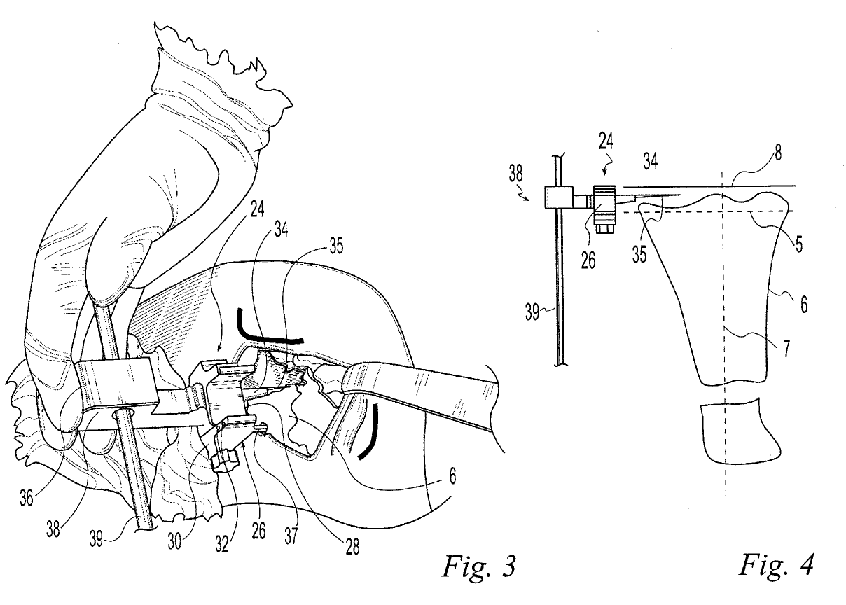

Once the lateral compartment 22 is formed, the tibial plateau of the tibia 6 is resected along a cut plane 5 that is substantially parallel to the joint line 8. In one embodiment, as depicted in FIG. 3, the proper orientation of the cut plane is determined by initially mounting a tibial template 24 on the anteriolateral side of the proximal end of the tibia 6.

The tibial template 24 comprises a base 26 having a front face 28 and an opposing back face 30. At least two spaced apart passageways 32 extend between the front 28 and back 30 faces. A placement arm 34 having a flat end 35 projecting outwardly from the front face 28 is spaced a predetermined distance from the passageways 32. A support arm 37 also projects from the front face 28 at the same distance from the placement arm and the passageways 32. The support arm and passageways are selectively adjustable along the height of the front face 28 to vary their spacing from the support arm 37. The support arm 37 thus indicates the location on the bone that fasteners will penetrate when inserted through the passageways 32.

Finally, an alignment assembly 38 is mounted on the back face 30 of the base 26. The alignment assembly 38 includes a bracket 36 that is rotatable relative to the base 26 about an axis that is substantially parallel with the longitudinal axis of the placement arm 34. An elongated rod 39 projects from the bracket 36 at an orientation substantially normal to the long axis of the placement arm 34.

The flat end 35 of the placement arm 34 is rested on the lower posterior side of the lateral facet of the superior articular surface of the tibia 6. The medial-lateral tilt (or varus-valgus angle) of the cut plane for resection of the tibial plateau is set by positioning the rod 39 in parallel alignment with the mechanical axis of the tibia 6. In one embodiment, this is accomplished by using line-of-sight to position the rod 39 in parallel alignment with the tibial ridge of the tibia 6, as shown in FIG. 4.

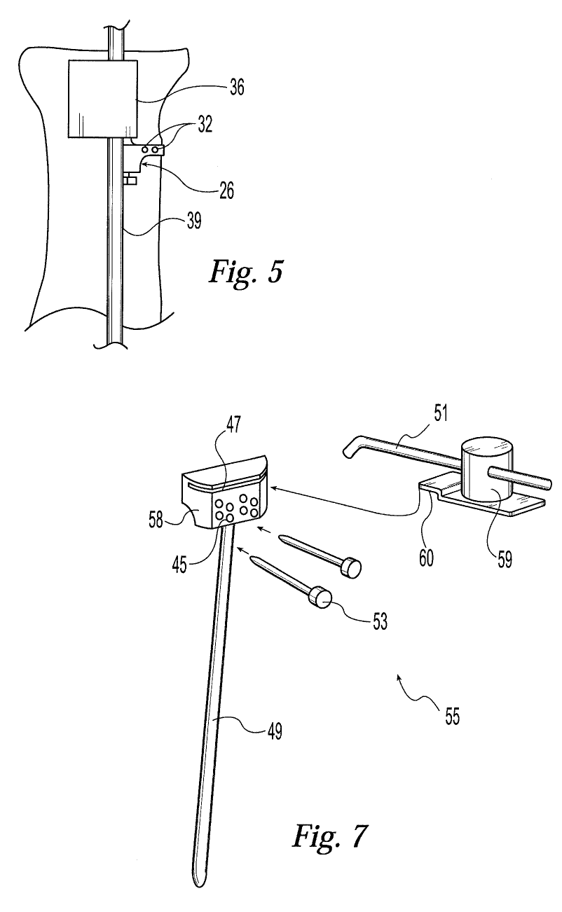

The anterior-posterior tilt (or posterior slope) for the cut plane is set by orienting the flat end 35 of the placement arm 34, which is rigidly mounted to the base 26, in alignment with the plane of the joint line 8. In one embodiment this is accomplished through feel and line-of-sight, as shown in FIG. 5. Alternatively, by using relative degree markings formed between the bracket 36 and base 26 of the tibial template 24, the base 26 and the placement arm 34 can be set at a predetermined angle relative to the rod 39 which, as discussed above, is disposed in parallel alignment with the mechanical axis of the tibia 6. The angle can be set in a range between about 3°. to about 7°. which is the statistical norm for the posterior slope of the joint line 8. Alternatively, the angle can be measured by using conventional instruments to measure the change in height between the anterior and posterior side of the superior articular surface.

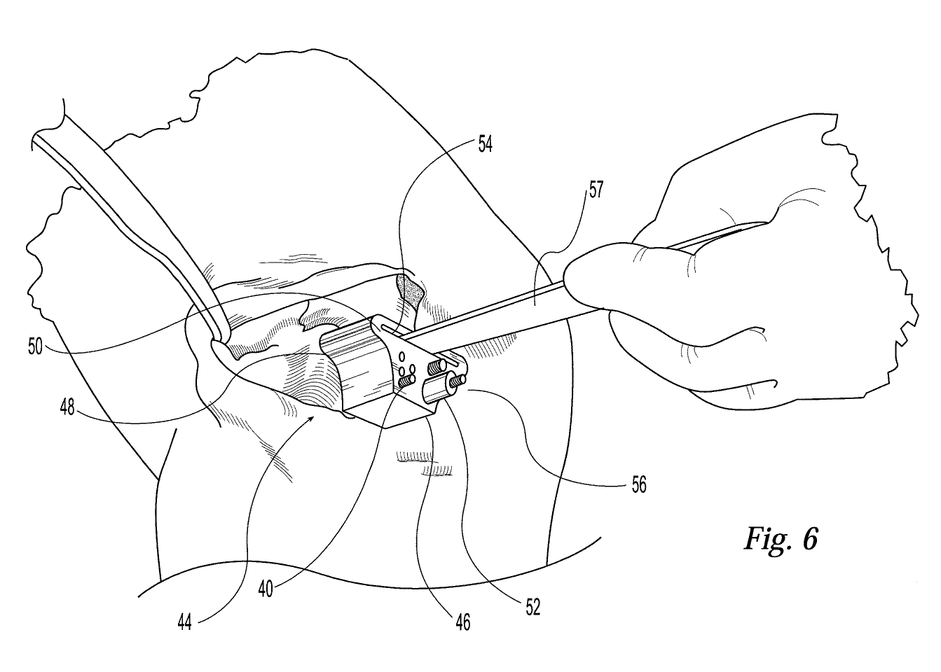

Once the tibial template 24 is properly oriented, fasteners 40 (see FIG. 6), such as pins, nails, screws, and the like, are drilled, hammered or otherwise passed through the passageways 32 and into the proximal end of the tibia 6 distal of the cut plane. Tibial template 24 is then removed over the fasteners 40 so that the fasteners 40 remain in place. The fasteners thus establish a datum for referencing the varus-valgus angle and posterior slope of the proximal tibial cut that records, or preserves, this position information after the template is removed.

As depicted in FIG. 6, a tibial guide 44 is then mounted on the fasteners 40. Specifically, the tibial guide 44 comprises a body 46 having a front face 48 and an opposing back face 50 with passageways 52 extending therebetween. The passageways 52 of the tibial guide 44 have substantially the same size and spacing as the passageways 32 of the tibial template 24. However, extra passageways 52 can be provided to allow for vertical fine tuning of the tibial guide 44. A bounded guide slot 54 also extends between the front 48 and back 50 faces. The tibial guide 44 is advanced so that the fasteners 40 are received within corresponding passageways 52. A threaded nut 56 or other form of retaining structure is then mounted on the exposed proximal end of each fastener 40 so as to tightly secure the tibial guide 44 to the tibia 6. As a result of the predetermined positioning of the fasteners 40, the guide slot 54 defines a cut plane at a predetermined location approximately 10 mm distal of the top of the tibial plateau.

A stylus 57 can be inserted through the guide slot 54 to visualize where the cut will be made to permit further adjustment if desired. If adjustment is necessary, the tibial guide 44 can be pulled off of the fasteners 40 and repositioned on a different set of passageways 52. An oscillating saw blade is then passed through the guide slot 54 and used to cut through the proximal end of the tibia 6 along the cut plane from lateral to medial. This cut is completed anteriorly and medially sacrificing the anterior cruciate ligament, but not posteriorly. Prior to removal of the tibial plateau, the bone is resected around the posterior cruciate ligament. In one embodiment, a ½-inch curved osteotome is inserted from the lateral side of the joint with the knee in varus to protect the bone block containing the tibial insertion of the posterior cruciate ligament. A large osteotome is then inserted into the cut and used to elevate the cut proximal tibial bone. The cut bone is put in traction by gripping it with a pair of forceps. The soft tissues are then removed from the periphery allowing extraction of the cut bone.

In an alternative embodiment, conventional arthroscopic procedures can be used to drill or otherwise resect the bone bounding the posterior cruciate ligament prior to initial cutting of the incision 20. Arthroscopic procedures can also be used to remove the meniscus and any soft tissue attachments to the proximal tibia that restrict the removal of the cut proximal tibia from the lateral side.

It is appreciated that there are a number of alternative methods and instruments that can be used in association with resection of the tibial plateau. For example, the tibial template 24 and tibial guide 44 can be combined into a single guide system that does not require changing parts over fasteners 40. FIG. 7 shows an illustrative embodiment of such a guide system 55. The guide system 55 comprises a base 58 through which passageways 45 and a guide slot 47 are formed. An elongated rod 49 projects from the base either rigidly or hingedly. A placement arm 51 is mounted on a bracket 59 having a flange 60 projecting therefrom.

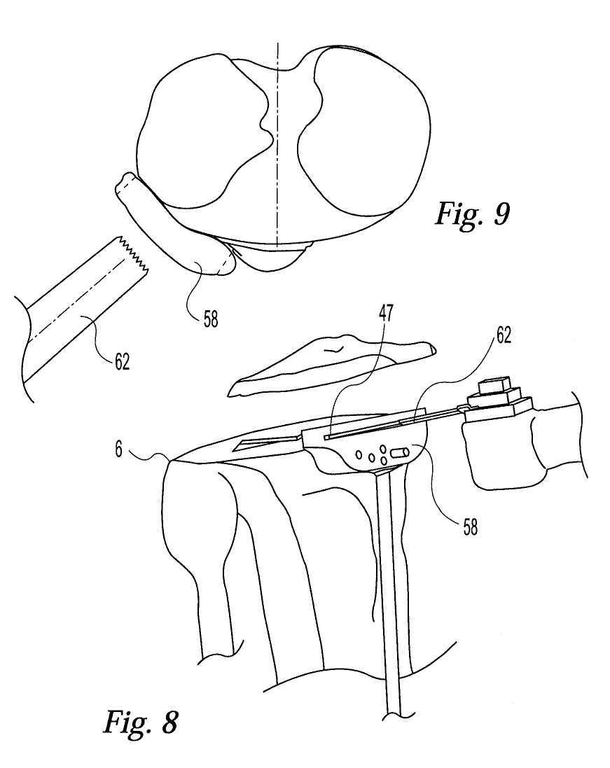

During initial attachment, the flange 60 is inserted into the guide slot 47 so that the base 58 can be properly oriented using substantially the same procedure as discussed above with regard to the tibial template 24. Once the base 58 is oriented, fasteners 53 are passed through the passageways 45 to secure the base 58 to the tibia 6. As depicted in FIGS. 8 and 9, the placement arm 51 is then removed from the base 58 so that a blade 62 can be inserted into guide slot 47. Alternatively, it is also appreciated that base 58 can be formed with a large exposed top surface which functions as a guide without the need for a bounded guide slot. Various instruments such as drills, oscillating chisels, oscillating saws, and other conventional bone cutting instruments can be used to remove the tibial plateau.

With the tibial plateau removed, the femur 4 and tibia can be moved together to allow more movement in the joint and give more room to access the femur. In this position, a cutting guide is mounted on the lateral side of the distal end of the femur 4 to facilitate selective resection thereof. The cutting guide, however, must be appropriately positioned so that each of the cuts, as discussed below, has a desired orientation.

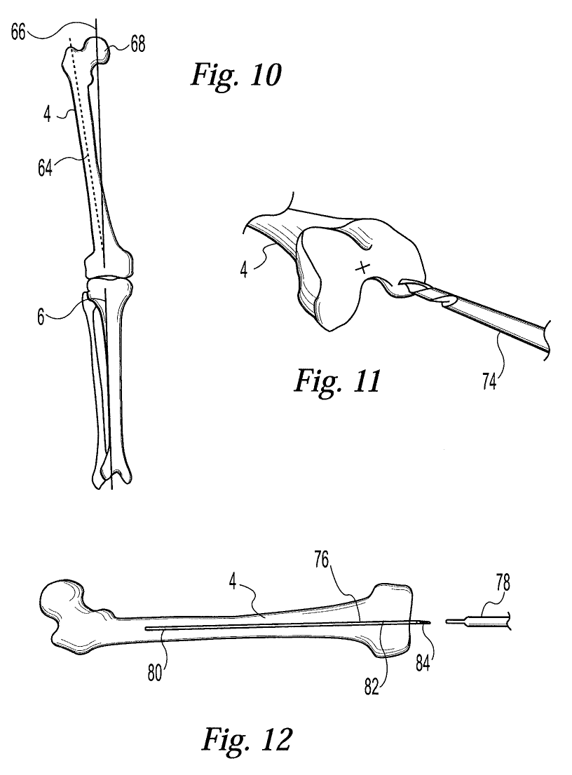

As depicted in FIG. 10, the femur 4 has associated therewith both an anatomic axis 64, which extends centrally along the femoral shaft, and a mechanical axis 66. The mechanical axis 66 defines the axis through which vertical load is carried by the femur 4. The mechanical axis 66 extends from the center of the femoral head 68 to the center of the distal end of the femur 4 at an angle approximately 6°. from the anatomic axis 64. The cuts on the distal end of femur 4 are made relative to the mechanical axis 66. As such, a reference to the mechanical axis 66 is first ascertained.

By way of example, in one embodiment an incision is made through the skin of the patient in the parapatella region such that the incision is in alignment with the anatomic axis 64. As depicted in FIG. 11, a guide wire 74 is passed through the incision and drilled into the distal end of femur 4 so as to extend into the medullary canal along at least a portion of the length of the anatomic axis 64. In one embodiment, the alignment of guide wire 74 with the anatomic axis 64 is established by using fluoroscopic observation simultaneously with drilling of the guide wire 74. Alternatively, a larger guide wire can be inserted into the medullary cannel and aligned with the anatomic axis 64 through sight and feel.

As depicted in FIG. 12, the guide wire, or intramedullary rod, 74 includes a main portion 76 and an extension portion 78. The main portion 76 includes a distal end 80 and an opposing proximal end 82. The proximal end 82 terminates at a threaded tip 84 which freely projects from the distal end of the femur 4. During placement, the extension portion 78 is threaded onto the main portion 76. A drill handpiece is then mounted to extension portion 78 for drilling the guide wire 74 into the femur. Once the guide wire 74 is inserted, extension portion 78 is temporarily removed. In this position, the knee and soft tissue are manipulated so that the proximal end 82 of the main portion 76 or the guide wire 74 is shifted from the incision formed in the parapatella region to the lateral compartment 22. The extension portion 78 is then reattached to the main portion 76.

As an alternative to making an incision in the parapatella region, the knee joint and soft tissue can initially be manipulated so that the guide wire 74 is drilled directly into the distal end of the femur 4 through the lateral compartment 22. In this embodiment, it is not necessary that the guide wire 74 be comprised of two portions.

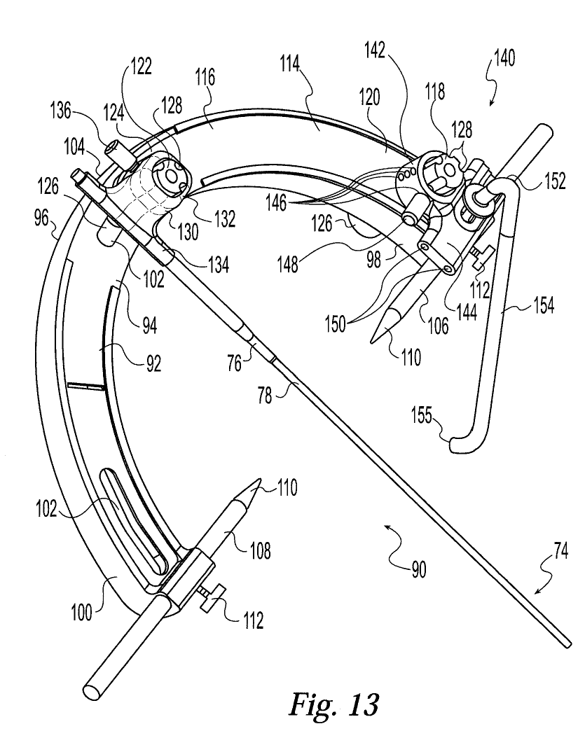

As depicted in FIG. 13, a positioning guide 90 is slidably attached to the guide wire 74. The positioning guide 90 comprises a substantially U-shaped body 92 having a top side 94 and a bottom side 96 each extending between a first end 98 and an opposing second end 100. Elongated slots 102 extend between the opposing sides 94, 96 at the first end 98, second end 100, and a central portion 104 of the body 92. A first guide stake 106 and a second guide stake 108 are slidably mounted on the ends 98, 100 of the body 92. Each guide stake 106, 108 has a pointed distal end 110. The guide stakes 106, 108 are disposed in coaxial alignment with their distal ends 110 facing oppositely. A knob 112 is associated with each guide stake 106, 108. Each knob 112 is threaded through a portion of the body 92 to selectively bear against its corresponding guide stake 106, 108 to enable selective fixed attachment of the guide stakes 106, 108 to the body 92.

A guide border 114, is positioned on the top side 94 of the body 92. The guide border 114 comprises an arched plate 116 having a first post 118 projecting upwardly from a first end 120 of the plate 116 and a second post 122 projecting upwardly from a second end 124 of the plate 116. A plurality of radially spaced apart grooves 128 are recessed along the side of each post 118, 122. Knobs 126 extend through the slots 102 at the first end 98 and central portion 104 of the body 92 and engage the posts 118, 122 so as to selectively secure the guide border 114 to the body 92. The knobs 126 can be loosened to allow the guide border 114 to slide in an arc along the body 92 to change the angle of the guide wire 74 relative to the guide stakes 106, 108. The knobs 126 can be tightened to fix the guide border in place. Depending on the operating parameters, the knobs 126 can be removed and the guide border 114 shifted to the second end 100 of the body 92.

A retainer 130 is mounted to the second post 122. The retainer 130 comprises a collar 132 that encircles the second post 122 and a sleeve 134 that encircles the guide wire 74. A set screw 136 is threaded through the collar 132 so as to bear against the second post 122 within a select groove 128. By loosening set screw 136, the retainer 130 can be selectively raised or lowered along the second post 122. When the set screw 136 is tightened, the engagement between the set screw 136 and corresponding groove 128 prevents rotation of the retainer 130 about the second post 122 and raising or lowering of the retainer 130 along the second post 122.

A pilot 140 is selectively mounted to the first post 118. The pilot 140 comprises a collar 142 that encircles the first post 118 and a pin guide 144 that projects outwardly from a side of the pilot 140. A pair of spaced apart channels 150 extend through the pin guide 144 in parallel alignment. A slot 152 is recessed into one end of the pin guide 144 between the channels 150. An elongated stylus 154 is selectively mounted within slot 152 and projects from it.

A plurality of ports 146 extend through the collar 142 so as to communicate with the first post 118. A set screw 148 is threaded into a select port 146 so as to bear against the first post 118 within a corresponding groove 128. Each port 146 is positioned at a unique predetermined radial position on the collar 142 such that by positioning the set screw 148 in a specific port 146, the pin guide 144, and thus the channels 150 therein, rotates to a predefined angle. In the embodiment depicted, nine ports 146 are provided each having a one degree variance. For example, if the set screw 148 is fixed in the number one port, the channels 150 are oriented at a one degree offset from perpendicular to the guide wire 74. When the set screw 148 is in the number nine port, the channels 150 are oriented at a nine degree offset from perpendicular to the guide wire 74.

During operation, the proximal end of the guide wire 74 is slid within the sleeve 134 of the retainer 130 so that the opposing ends 98, 100 of the positioning guide 90 are positioned laterally and medially of the knee 10, respectively. A small stab wound is made over the medial epicondyle and the guide stakes 106, 108 are advanced so as to bear against the lateral epicondyle and medial epicondyle, respectively. To enable placement of the stakes 106, 108 over the respective epicondyles, it may be necessary to loosen the set screw 136 and slide the retainer 130 along the second post 122. It may also be necessary to loosen the knobs 126 and slide the plate 116 along the arc defined by the U-shaped body 92 thereby changing the angle of the guide wire 74 relative to the guide stakes 106, 108. Once the stakes 106, 108 are appropriately positioned, the knobs 112, 126 and set screw 136 are tightened so that the stakes 106, 108 and retainer 130 are locked in place. In this position, the guide wire 74 is still disposed in alignment with the anatomic axis of the femur 4 and the guide stakes 106, 108 lie along the epicondylar axis. The epicondylar axis will be used to establish the external rotation for subsequent femoral cuts and implant placement.

The pilot 140 is set at an angle corresponding to the angle between the mechanical axis and the anatomic axis of the femur either before or after attachment of the positioning guide 90 to the guide wire 74. In one embodiment, this angle is determined in a preoperative procedure by use of standing x-ray. Alternatively, the angle can be set to approximately 6°. which is a statistical norm. Again, the desired angle is set by inserting the set screw 148 into a corresponding port 146 on the first post 118 so that the set screw 148 is received within a corresponding groove 128. This effectively sets the varus-valgus angle for subsequent bone cuts and implant placement.

Finally, the pilot 140 is also set at a desired anterior-posterior position along the length of the first post 118. This is set by raising or lowering the pilot 140 along the first post 118 until the free end 155 of the stylus 154 contacts the surface of the lateral anterior femoral ridge. This determination is made by sight and feel. Once the pilot 140 is at the proper orientation, it is secured in place by tightening the set screw 148. This effectively sets the anterior-posterior position for subsequent bone cuts and implant placement.

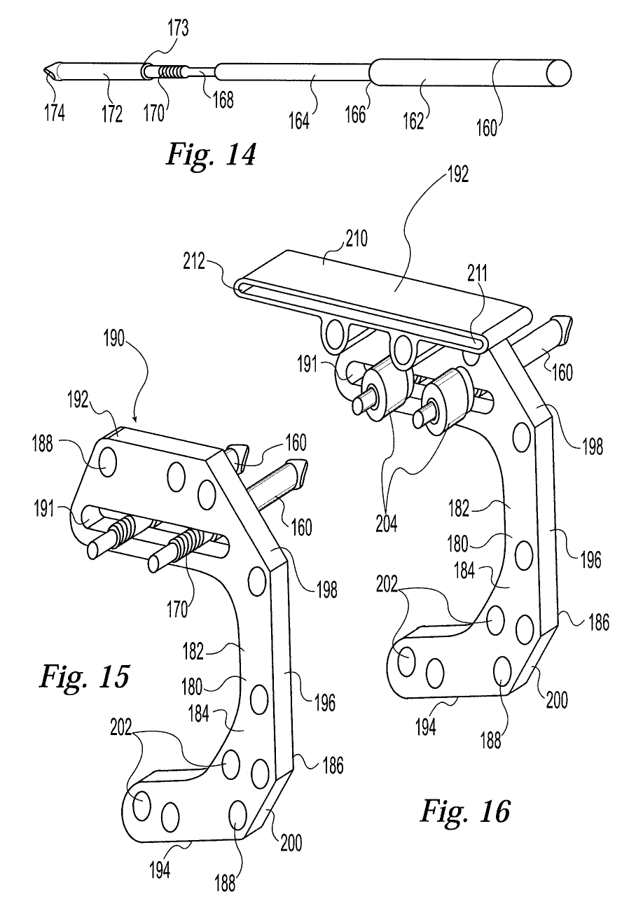

Once the positioning guide 90 is locked in place, alignment pins 160 are passed through the channels 150 of the pilot 140 and drilled, hammered or otherwise advanced into the lateral side of the femur 4. As depicted in FIG. 14, each alignment pin 160 has a proximal segment 162 and an intermediate segment 164 having a diameter smaller than the diameter of the proximal segment 162. As such, a proximal annular shoulder 166 is formed between the proximal segment 162 and the intermediate segment 164. A narrow breakaway segment 168, a threaded segment 170, and a distal segment 172 terminating at a sharpened tip 174 extend distally of the intermediate segment 164. The threaded segment 170 is smaller in diameter than the distal segment 172 thereby forming a distal annular shoulder 173 between the distal segment 172 and the threaded segment 170. The alignment pins 160 are advanced until the shoulders 166 contact the pilot 140, thereby precluding further advancement and placing the two pins at the same depth relative to the pin guide 144. In this position, as shown in FIG. 11, the distal tips of the alignment pins 160 are disposed adjacent to the guide wire 74.

Once the alignment pins 160 are placed, the positioning guide 90 is loosened and removed from the knee 10. The positioning guide 90 is separated from the implanted pins 160 by fracturing each pin 160 at the breakaway segment 168. The guide wire 74 is also removed from the femur 4. The two alignment pins 160 now act as datums and record by their placement the desired varus-valgus angle, external rotation angle, and anterior-posterior position as determined during the placement of positioning guide 90.

As depicted in FIG. 15, a template 180 is mounted on the alignment pins 160. The template 180 comprises a substantially U-shaped body 182 having an exposed perimeter edge 190 comprised of a plurality of flat planar surfaces. Specifically, the perimeter edge 190 comprises an anterior surface 192, an opposing posterior surface 194, a distal surface 196, a first chamfered surface 198 extending between the anterior 192 and distal 196 surfaces, and a second chamfered surface 200 extending between the posterior 194 and distal 196 surfaces. In alternative embodiments, it is appreciated that the body 182 need not be U-shaped but can be any substantially square or any other desired configuration that contains the desired surfaces on perimeter edge 190.

Body 182 further includes a front face 184, a back face 186, and a plurality of spaced passageways 188 extending between them. In one embodiment at least two passageways 188 are disposed adjacent to each surface of the perimeter edge 190, although a single passageway 188 can be associated with more than one surface. An elongated slot 191 and one or more anchoring ports 202 also extend between the front 184 and back 186 faces.

As depicted in FIG. 16, the template 180 is mounted on the alignment pins 160 by passing slot 191 over the pins 160 and then threading nuts 204 onto the exposed threaded segments 170 of each pin 160. The nuts 204 bias the template 180 against the distal annular shoulder 173 of each pin 160 so as to secure the template 180 in place and oriented in the varus-valgus and posterior slope alignment pre-set by the pins. In this position, the template 180 is mounted on the lateral side of the femur 4 with the surfaces 192, 194, 196, 198, 200 of the perimeter edge 190 denoting cut planes for the femur 4. By measuring the distance between each surface of perimeter edge 190 of the template 180 and corresponding outer surfaces of the femur 4, a template 180 of an appropriate size is selected so that the cuts on the femur 4 are made at the desired thickness. By loosening the nuts 204, the template 180 can be slid on the pins to adjust the proximal-distal position for subsequent bone cuts and implant placement. Once an appropriately sized template 180 is chosen, positioned, and secured on the alignment pins 160, additional pins, anchors, screws or other types of fastens are anchored into the femur 4 through the anchoring ports 202, thereby further securing template 180 to the femur 4.

A modular cut guide 210 is selectively mounted on the front face 184 of the template 180. The cut guide 210 has a bounded guide slot 212 and a pair of spaced apart passageways 214 extending through it. The cut guide 210 is configured such that its passageways 214 can be selectively aligned with each pair of passageways 188 associated with each perimeter edge surface of the template 180. Screws can be passed through the cut guide passageways 214 and threaded into the template passageways 188 to secure the cut guide 210 to the template 180. Alternatively, any form of fastener such as screws, nails, pins and the like can be passed through both the cut guide 210 and the template 180 and secured within the femur 4, thereby also securing the cut guide 210 to the template 180. By securing the cut guide 210, a bottom surface 211 of the guide slot 212 is disposed in the same plane as the corresponding surface of the perimeter edge 190 of the template 180. A saw, drill, chisel or the like is then passed through the guide slot 212 so as to resect the distal end of the femur 4 along the cut plane defined by the guide slot 212 and the corresponding surface of the perimeter edge 190 of the template 180. Once a cut is completed, the cut guide 210 is moved into alignment with the next perimeter edge surface of the template 180 and another cut is made. As such, each cut is made individually beginning anteriorly and extending distally and posteriorly.

It is appreciated that the cuts can be formed on the femur 4 using a number of different techniques and apparatus. For example, instead of the cut guide 210 having the bounded slot 212, a cut guide can be provided which simply provides an enlarged exposed support surface that is disposed in the same plane as the perimeter edge surfaces of the template 180. The cutting instrument, which can comprise any form of drill, blade, chisel or the like is then supported on the support surface while facilitating the cuts. In like manner, a separate cut guide can be eliminated and simply replaced with a thicker template which also functions as the cut guide. Likewise, the template need not have a cut guiding surface on its perimeter. The cut guide slot alone can guide the cut.

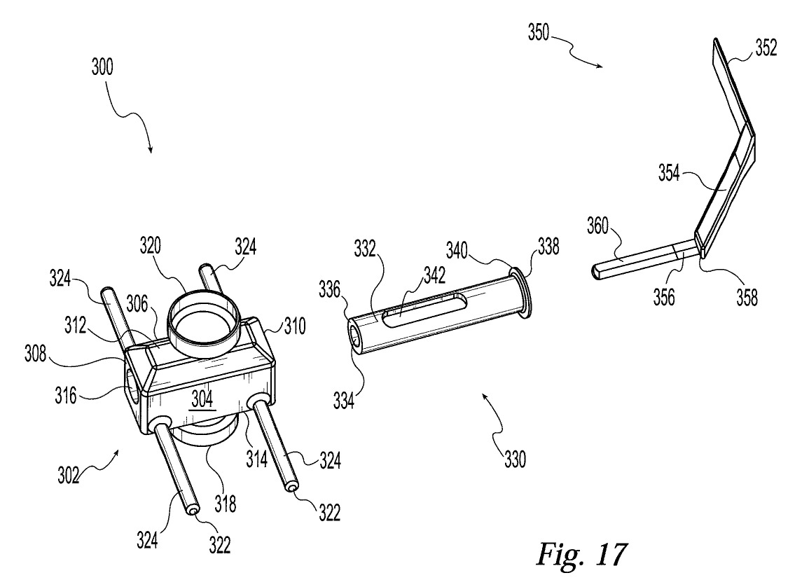

FIGS. 17-21 depict an alternative illustrative embodiment of a template and modular cut guide assembly for use with the alignment pins 160 set in the femur. Referring to FIG. 17 the template 300 comprises a distal cut guide body 302 having opposing first 304 and second 306 side walls, opposing first 308 and second 310 end walls, and opposing top 312 and bottom 314 faces. An axial through bore 316 extends through the cut guide body 302 from the first end wall 308 to the second end wall 310. A slide lock knob 318 has a threaded shaft (not shown) threaded into a bore in the bottom face 314 in communication with the axial through bore 316. An implement lock knob 320 has a threaded shaft (not shown) threaded into a bore in the top face 312 in communication with the axial through bore 316. A pair of pin receiving bores 322 extends through the cut guide body 302 transverse to the axial bore 316. The pin receiving bores 322 are offset from the axial bore 316 so that pins may pass through them without interfering with the axial bore 316. The length of each pin receiving bore 322 is extended by a pin sleeve 324 projecting from each side 304, 306 of the cut guide body 302.

A slide 330 comprises a cylindrical body 332 having an axial through bore 334 extending from a first end 336 to a second end 338. An annular flange 340 extends radially from the second end 338 and has a diameter larger than the axial through bore of the cut guide body 302. An elongated slot 342 extends through one side of the slide body 332 in communication with the through bore 334. The slide 330 is slidably received in the through bore 316 of the cut guide body 302. Slide lock knob 318 can be threaded further into the cut guide body 302 to bear against the slide 330 and lock it in a desired axial position along the through bore 316 axis. The flange 340 will bottom on the second end wall 310 to prevent the slide 330 from sliding completely through the cut guide body 302. The shaft of the implement lock knob 320 aligns with the slot 342 when the slide is inserted into the cut guide body 302.

An implement 350 includes a working end 352, a support arm 354, and a mounting shaft 356. The base of the arm 354 is larger than the mounting shaft 356 diameter such that a shoulder 358 is formed at the junction of the shaft 356 and arm 354. The shaft 356 is generally cylindrical with a flat 360 formed along one side. The mounting shaft 356 is slidably received in the axial through bore 334 of the slide 330. The shoulder 358 will bottom on the second end 338 of the slide such that the working end 352 is located at a predetermined distance from the second end 338. The implement lock knob 320 can be threaded further into the cut guide body 302 and through the slot 342 in the slide 330 to bear against the slide flat 360 and lock the shaft in a desired axial position along the slide through bore 334 axis. A selection of implements 350 is provided offering different working ends 352. Each implement in the selection has a mounting shaft for engaging the slide 330 through bore 334 and shoulder for bottoming on the second end 338 to position its working end at a predetermined distance from the second end 338 of the slide.

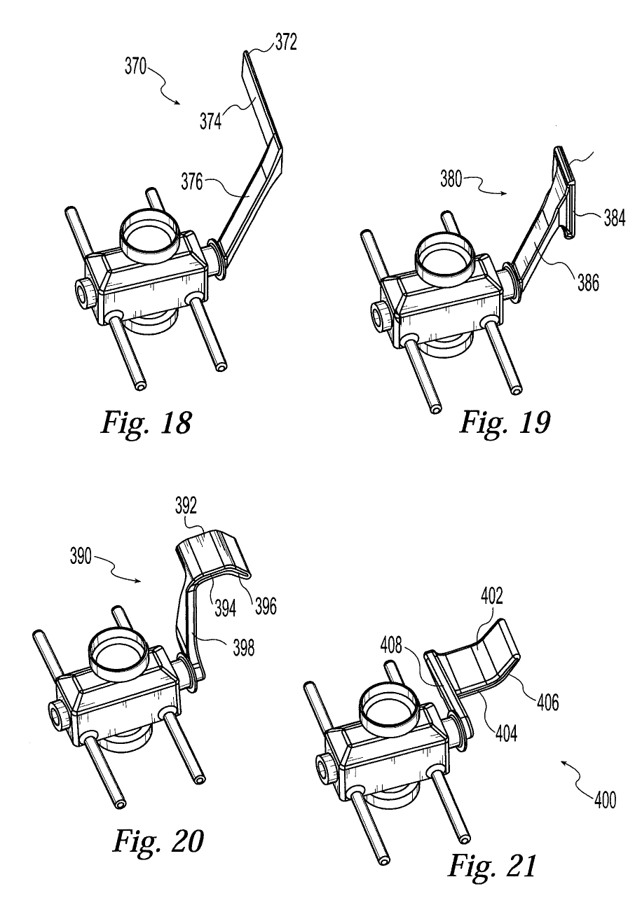

FIGS. 18-21 depict a progression of implements mounted on the cut guide body for preparing the distal femur. A distal cut stylus 370 has a flat working end 372 defining a reference surface 374. The working end 372 connects via a support arm 376 to a shaft (not shown) forming a shoulder as described in reference to FIG. 18. The reference surface 374 lies at a known distance and orientation relative to the shaft and shoulder. In the illustrative embodiment, the reference surface 374 lies in a plane generally perpendicular to the shaft at a predetermined distance from the shoulder. The reference surface 374 projects away from the shaft axis toward the femoral bone to position the reference surface 374 in the incision adjacent to the bone.

A distal femoral cut guide 380 has a cut guide working end 382 for guiding a cutter to cut the distal femur. In the illustrative embodiment, the working end 382 comprises a saw slot 384 for maintaining a saw blade in a desired cutting plane. The working end 382 connects via a support arm 386 to a shaft (not shown) forming a shoulder as previously described. The saw slot 384 lies at a known distance and orientation relative to the shaft and shoulder. In the illustrative embodiment, the saw slot lies in a plane generally perpendicular to the shaft at a predetermined distance from the shoulder. The working end 382 projects away from the shaft axis toward the femur to position the saw slot in the incision adjacent to the bone.

An anterior femoral cut guide 390 has a cut guide working end 392 for guiding a cutter to make the anterior and anterior chamfer cuts on the distal femur. In the illustrative embodiment, the working end 392 comprises an anterior cut saw slot 394 and an anterior chamfer cut saw slot 396. The working end 392 connects via a support arm 398 to a shaft (not shown) forming a shoulder as previously described. The saw slots 394, 396 lie at known distances and orientations relative to the shaft and shoulder. The working end 392 projects away from the shaft axis toward the femur to position the saw slots in the incision adjacent to the bone.

A posterior femoral cut guide 400 has a cut guide working end 402 for guiding a cutter to make the posterior and posterior chamfer cuts on the distal femur. In the illustrative embodiment, the working end 402 comprises a posterior cut saw slot 404 and a posterior chamfer cut saw slot 406. The working end 402 connects via a support arm 408 to a shaft (not shown) forming a shoulder as previously described. The saw slots 404, 406 lie at known distances and orientations relative to the shaft and shoulder. The working end 402 projects away from the shaft axis toward the femur to position the saw slots in the incision adjacent to the bone.

In use, alignment pins 160 are placed in the femur as described above to establish desired varus-valgus angle, external rotation angle, and anterior-posterior position. The pin receiving bores 322 of the cut guide body 302 are slid over the alignment pins 160 until one or both of the pin sleeves 324 bottoms on the femoral bone. A nut can be threaded onto each of the alignment pins 160 to secure the cut guide body in place. The distal cut stylus 370 is then inserted into the slide 330 until its shoulder bottoms on the second end 338 of the slide 330. The slide 330 is translated axially within the cut guide body 302 through bore 316 until the reference surface 374 contacts the lateral distal condyle. With the distal cut stylus 370 bottomed in the slide 330 the slide lock knob 318 is rotated to lock the slide 330. This fixes the proximal-distal position of all of the femoral cuts since each cut guide is referenced to the end 338 of the slide 330. Now the varus-valgus angle, external rotation angle, anterior-posterior position, and proximal-distal position of each cut are now fixed since the cut guides are keyed to the slide 330, which is locked to the cut guide body, which is in turned fixed in position by the alignment pins through the pin receiving bores 322. The distal cut stylus 370 is removed from the slide 330. The distal cut guide 380 is slid into the slide 330 until it bottoms and is locked in place by tightening the implement lock knob 320. A saw blade is directed through the saw slot 384 to resect the distal femur. The anterior 390 and posterior 400 femoral cut guides are used similarly to make the anterior, anterior chamfer, posterior and posterior chamfer cuts. The anterior 390 and posterior 400 femoral cut guides can be provided in a range of sizes to prepare different sizes of femurs to receive appropriately sized implants.

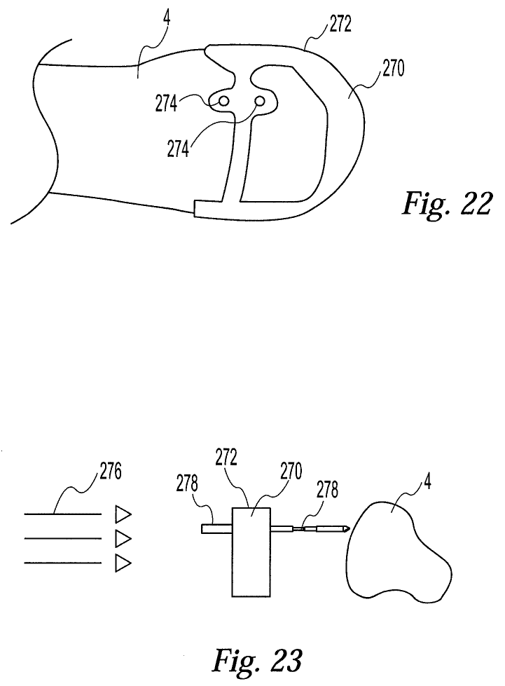

In an alternative embodiment, templates can be mounted on the lateral side of the femur 4 without the use of the positioning guide 90. FIG. 22 depicts a set guide 270 having an exposed perimeter surface 272. The perimeter surface 272 has a configuration substantially complementary to the perimeter contour of the distal end of the femur 4 taken from a lateral view. A pair of spaced apart passageways 274 extends through the set guide 270. In one embodiment, the set guide 270 is comprised of a material that is semitransparent to fluoroscopic rays.

As depicted in FIGS. 22 and 23, the set guide 270 is disposed adjacent the lateral compartment 22 so that the perimeter surface 272 of the set guide 270 is aligned with the perimeter contour of the distal end of the femur 4 taken from a lateral view. In one embodiment, this alignment is assisted by the use of real-time fluoroscopic rays 276 passing through the set guide 270 and the femur 4. Once the set guide 270 is aligned with the distal end of the femur 4, fasteners 278 are passed through each of the passageways 274 and into the femur 4. In one embodiment, the fasteners 278 can comprise the alignment pins 160. Once the fasteners 278 are placed, the set guide 270 is removed. The template is then mounted on the fasteners 278 in the same way that the template is mounted on the alignment pins 160. The cutting process is then completed in the same way as previously discussed with regard to the template.

Once the cuts are made and the bone fragments are removed through the lateral compartment 22, the template, the cut guide 210 and all related pins and fasteners are removed from the femur 4. The distal end of the femur 4 is moved laterally to expose the femur through the lateral incision 20. Conventional cuts are then made on the posterior side of the patella to accommodate a prosthetic patellar articular surface. All of the prosthetic components can then be fixed in place through the lateral incision.

Closure is obtained by leaving the retinaculum open on the lateral side adjacent to the patella and closing only the retinaculum beginning at the proximal and lateral patella and extending distally to the tibial tuberosity. Gerdy's tubercle does not have to be reattached, because this has been partially excised in the excision of the proximal tibia, and the iliotibial band is continuous with the aponeurosis over the perineal musculature. It will reattach itself and secure anterolateral stability.

The above described minimally invasive process for total knee arthroplasty is described with reference to forming incision 20 and thus compartment 22 on lateral side 12 of knee 10. It is appreciated that the same methods and instruments can be used to perform the minimally invasive procedure on the medial side of knee 10. The described embodiments are to be considered in all respects only as illustrative and not restrictive. The present invention may be embodied in other specific forms without departing from the spirit and scope of the appended claims.

1-21. (canceled)

22. A femoral positioning guide for establishing a position reference on a femur to guide the cutting of the distal femur, the femur having a proximal end, a distal end, an anatomic axis from the proximal end to the distal end, an intramedullary canal along the anatomic axis, a medial epicondyle, a lateral epicondyle, an anterior surface and a posterior surface, the femoral positioning guide comprising:
a body having a closed end and an opposite open end with opposing sides able to straddle the distal end of the femur;
a pair of probes mounted on the opposing sides in coaxial alignment, the pair of probes being simultaneously alignable with the medial and lateral epicondyles;
an intramedullary rod mounted on the body, the intramedullary rod being engageable with the intramedullary canal; and
means for establishing a position reference on the femur that records a desired position and orientation in a way that permits positive engagement by a bone cutting guide to produce a bone cut relative to the position and orientation information.

23. The femoral positioning guide of claim 22 wherein the means for establishing a position reference on the femur is a pin guide mounted on the body, the pin guide having at least one passageway for guiding the placement of an alignment pin in the femur.

24. The femoral positioning guide of claim 23 wherein the pin guide is rotatably mounted on the body so that the pin guide can be rotated to a desired varus-valgus angle relative to the intramedullary rod.

25. The femoral positioning guide of claim 24 wherein the pin guide is also mounted for translation from a position nearer the body to a position further from the body, the pin guide further including a stylus able to reference the anterior surface of the femur to set the pin guide at a desired translated position relative to the body.

26. The femoral positioning guide of claim 25 wherein the body defines a circular arc, the positioning guide further comprising a plate translatably mounted on the body, the plate able to be translated along a portion of the arc, the pin guide and the intramedullary rod both being mounted on the plate so that they can be angled relative to the probes by translating the plate along the arc of the body while remaining in fixed angular relation to one another.

27. A method comprising the steps of:
positioning a femoral position guide adjacent a distal end of a femur, the femoral position guide having a position reference guide able to be positioned at a desired varus-valgus angle and at a desired external rotation angle;
determining a desired varus-valgus angle and a desired external rotation angle;
creating a position reference on the femur that permits positive engagement by a cut guide to orient the cut guide at the desired varus-valgus and external rotation angles;
engaging a cut guide with the position reference; and
guiding a cutter with the cut guide to cut the femur.

28. The method of claim 27 wherein the step of creating a position reference on the femur comprises inserting two reference pins into the femur with a pin guide.

29. The method of claim 28 further comprising the step of engaging the medial and lateral epicondyles of the femur to orient the pin guide at a desired external rotation angle.

30. The method of claim 29 further comprising the step of inserting an intramedullary rod into the intramedullary canal of the femur and angling the pin guide relative to the intramedullary rod to orient the pin guide at a desired varus-valgus angle.

31. The method of claim 30 further comprising the step of referencing the anterior femoral surface with a probe associated with the pin guide to position the pin guide at a desired anterior-posterior position.

32-70. (canceled)
